(19) United States
(12) Patent Application Publication
(10) Pub. No.: US 2016/0074353 A1
(43) Pub. Date: Mar. 17, 2016

(54) USE OF PETROSELINIC ACID TO FIGHT AGAINST AESTHETIC DISORDERS OF THE BODY FIGURE

(71) Applicants: L'OREAL, Paris (FR); NESTEC SA, Vevey (CH)

(72) Inventors: Yann MAHE, Sainte Geneviève des Bois (FR); Carole BRU, Courbevoie (FR); Audrey GUENICHE, Rueil Malmaison (FR)

(21) Appl. No.: 14/890,064

(22) PCT Filed: May 7, 2014
(86) PCT No.: PCT/IB2014/061264

§ 371 Date: Nov. 9, 2015

(30) Foreign Application Priority Data
May 7, 2013 (FR) 13 54186

(51) Int. Cl. A61K 31/201; A61K 31/315; A61K 31/01; A61K 31/145

(52) U.S. Cl. CPC A61K 31/201; A61K 31/145; A61K 31/315; A61K 31/01

(57) ABSTRACT

The present invention concerns the cosmetic use, by oral administration, of petroselinic acid or of a combination of active ingredients comprising at least petroselinic acid and at least one compound chosen from zinc, taurine, one of the salts of same, lycopene and the mixtures thereof, and preferably at least taurine or zinc gluconate and, more preferably still, at least taurine and zinc gluconate, with the aim of fighting against aesthetic disorders of the body figure linked to modifications in the adipose tissue.

The present invention relates to the field of cosmetic compositions, including food supplements for combating esthetic disorders of the figure associated with adipose tissue modifications.

In particular, the invention is directed toward proposing the use of petroselinic acid or of combinations comprising at least petroselinic acid, which are useful for improving and/or slimming down the figure and improving the quality of adipose tissue. The quality of adipose tissue is especially impaired by the presence of cellulite or of the orange peel syndrome or else is affected when there is a change, in particular an abrupt change, in a person's weight, upward or downward.

Human skin consists of three compartments, namely a superficial compartment, which is the epidermis, the dermis and a deep compartment, which is the hypodermis. The latter compartment consists essentially of a type of cells that are specialized in the accumulation and storage of fats, the adipocytes.

In overweight individuals, and more particularly during weight gain, the adipocytes have a tendency to rapidly increase in volume (storage of increasing amounts of lipids). The fat lobules then distend little by little so as to result in the formation of connective trabeculae, parallel to one another and perpendicular to the skin surface. The strong pressure exerted by the adipocytes on the dermis rapidly causes a deformation of the surface of the skin.

In cutaneous terms, this phenomenon known as cellulite is reflected by a padded appearance giving, in places, signs of skin resembling an orange peel.

Finally, from the clinical point of view, cellulite is reflected by a modification of the texture of the subcutaneous and superficial tissues, characterized in particular by:
skin which, overall, is thicker,
skin which is more consistent,
skin which is more sensitive, and which may even, depending on the stage of progression of the cellulite, be painful on palpation, and/or
cutaneous tissues which are less mobile due to the loss of adhesion and of cohesion of the deep layers of skin.

Furthermore, this phenomenon is more visible in women since they have finer skin with connective trabeculae exhibiting a vertical structure, which, conversely, in men have an oblique and criss-cross structure.

Cellulite, which is often worsened by excess weight and obesity, is especially located around the pelvis and the lower limbs (“jodhpur thigh” or “zouave pants” cellulite). These modifications may also result in permanent scar deformations, especially in areas most subject to variations in adipose tissue mass, such as the buttocks, the hips, the stomach or the top of the legs.

Hypertrophy of the adipose tissue, associated especially with weight gain, is accompanied, at the dermal level, by the fiber network being placed under tension, leading to a functional modification of the resident cells. Indeed, this hypertension impedes cellular exchanges and venous and lymphatic circulation by compression of capillaries, so that the phenomenon is self-maintaining. In the end, the fibers degenerate and the skin loses its fundamental structures. The occurrence of an uneven (“orange peel”) skin, of more or less flaccid or gelatinous consistency, may also gradually give the figure an unsightly general appearance.

Furthermore, the development of the adipose mass can evolve between simple local excess weight (lipodysmorphia) and the formation of cellulite, passing through a certain level of stoutness and ending at actual obesity. Obesity is, on the other hand, a real disabling pathological condition when it results in particular in the development of a metabolic syndrome that the combination which is the subject of the invention does not, however, intend to treat since it addresses only esthetic disorders of the figure which are not associated with a pathological condition.

Similarly, consequences are, actually, visible at the surface of the skin also during weight loss which is sometimes too fast and in particular during slimming diets. Indeed, the rapid destorage of the adipocytes leads to a considerable decrease in the tension exerted by the hypodermis on the supporting tissues. When these variations in weight gain and weight loss follow each other, this is referred to as the “yoyo” effect. The appearance of cicatricial marks, also called stretch marks, which are the sign that the dermal tissue has been affected and has not been able to adapt to these overly large and overly rapid changes in adipose mass in the underlying hypodermis, may sometimes follow.

In biological terms, an inflammatory state of the adipose tissue may be observed during adipose tissue hypertrophy. Inflammation of the adipose tissue, in particular of the subcutaneous white adipose tissue (ScWAT), has been particularly well described in the case of obesity. Indeed, when the energy balance of the body is unbalanced, either through a lack of physical exercise or through excessive consumption of food (or both), the subcutaneous adipose tissue expands and accumulates under the skin. When this significant development of the adipose mass is maintained, a more general metabolic imbalance may follow. The adipose tissue is in fact considered, as a whole, to be an important endocrine organ, the physiology of which may be impaired by adipose cell hypertrophy and the accumulation of periadipocyte immune cells, in particular including macrophages.

It has thus been described that the pre-adipocytes of non-obese women respond to the factors produced by these macrophages and produce molecules and chemokines such as IL-8 and MCP1 which further amplify and maintain the adipose tissue inflammation by recruiting further inflammatory cells into the adipose tissue (D. Lacasa et al. Endocrinology 148 (2):868-87 (2007); M. Keophiphat et al. Molecular endocinology 23:11-24 (2009).

As an expected and ubiquitous physiological consequence of such a chronic inflammatory state associated in particular with an overly large development or else with an overly fast variation in size of the adipose tissue (both upward and downward), a “pro-fibrotic” phenotype may then develop in the inflammatory adipose tissue. When this pro-fibrotic phenotype develops more particularly in the subcutaneous white adipose tissue (scWAT), it contributes to the setting in of observable signs of modification at the actual skin surface, which is in particular cellulite.

Indeed, among many identified etiological factors, cellulite appears to be due to structural, morphological, inflammatory and biochemical modifications of the subcutaneous adipose tissue (Khan et al. Treatment of cellulite; part II advances and controversies J. Am. Acad. Dermatol. 373-384 (2010)). Macroscopically, cellulite is characterized by a moderate increase in the thickness of the dermis (+18%), and especially of the hypodermal adipose tissue (×3.2) and by strong indentations of the adipose tissue as far down as the dermis (B. Querleux; Cellulite; pathophysiology and treatment; Taylor and Francis group London Chapter 6, pages 105-113 (2006)). Fibrotic shapes are expected consequences of the inflammatory modifications of the subcutaneous adipose tissue, and are the result of a deep rearrangement of the peri-adipocyte tissue: women who have cellulite thus show a greater number of fibrous septae perpendicular to the surface of the skin in comparison with the subcutaneous adipose tissue of women without cellulite (p<0.001; B. Querleux et al. Skin Research and Technology; 8: 118-124 (2002)).

As illustrated in the examples of the present application, the inventors have noted that petroselinic acid or a combination of active agents in accordance with the invention proves to be capable of synergistically increasing the amount of lipoxin A4. Lipoxin A4 belongs to the resolvin family. This family of compounds naturally produced by the body acts in a manner complementary to conventional anti-inflammatory agents by raising the threshold for triggering a “dermatologically conventional” inflammatory response, and more particularly so as to raise the threshold of appearance of the signals of this conventional inflammation, namely redness, pain and heat.

Consequently, lipoxin A4 appears to be a potential target for acting on these skin parameters.

Consequently, the present invention is more particularly focused on identifying active agents or combinations of active agents that exert a significant action on lipoxin A4.

The use of petroselinic acid for preparing a composition intended for activating the peroxisomal β-oxidation of fatty acids in the superficial tissues of a mammal, so as to be able to treat or prevent inflammations and/or modulate lipid metabolism in these superficial tissues, is known from document EP 888 773. The skin conditions more particularly targeted in said document are inflammations associated with psoriasis, erythema (sunburn), eczema, seborrheic dermatitis, alopecia areata, mycosis, acne or other forms of dermatosis.

Thus, none of the prior art documents known to the inventors suggests that petroselinic acid or a combination of active agents comprising at least petroselinic acid and at least one compound chosen from zinc, taurine, a salt thereof, and lycopene, exerts activity on lipoxin A4.

A first subject of the invention is thus the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for the purposes of combating effects, especially non-pathological effects, on adipose tissue, and in particular for combating esthetic disorders of the figure associated with adipose tissue modifications.

A subject of the invention is also the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for reducing the weight of fat mass of an individual.

According to the present invention, the term “fat mass” is intended to denote the mass of adipose tissue, or fat, in an individual, as opposed to the muscle mass.

A subject of the invention is also the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for reducing the total weight of an individual.

A subject of the invention is also the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for limiting the expansion of and/or reducing adipose tissue, especially subcutaneous adipose tissue.

A subject of the invention is also the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for improving the figure by limiting or reducing, in particular, unsightly accumulations of subcutaneous adipose tissue on the waist, the hips, the thighs, the tummy, the arms and the face.

A subject of the invention is also the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for combating cellulite.

In particular, a subject of the invention is the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for preventing and/or treating the visible manifestations of cellulite and/or the orange peel appearance of the skin and/or else for treating stretch marks or for preventing their appearance.

A subject of the invention is also the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for the purposes of combating the signs of modification of the skin surface during an upward or downward variation in weight, in particular during weight loss especially observed in the course of a slimming diet or during weight loss observed prior to, simultaneously with and/or subsequent to a cosmetic surgery procedure, most particularly during weight loss observed in the course of a slimming diet.

The invention is also directed toward the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for maintaining and/or restoring the biomechanical properties of the skin, in particular chosen from the extensibility, tonicity, firmness, suppleness, density and/or elasticity properties of the skin.

In particular, the invention relates to the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for maintaining and/or restoring the extensibility, tonicity, firmness, suppleness, density and/or elasticity properties of the skin.

The invention is also directed toward the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for preventing and/or combating skin disorders induced by weight loss especially observed in the course of a slimming diet.

The invention is also directed toward the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, as an agent for firming the skin of an individual who has undergone a weight modification or prior to, simultaneously with and/or subsequent to a cosmetic surgery procedure.

The invention is also directed toward the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for preventing and/or combating skin disorders observed especially during excessive weight gain.

The invention is also directed toward the oral cosmetic use of petroselinic acid, or of a combination of active agents comprising at least petroselinic acid and at least one compound chosen from zinc, taurine, a salt thereof, lycopene, and mixtures thereof, and preferably at least taurine or zinc gluconate, and even more preferably at least taurine and zinc gluconate, for preventing and/or combating the expansion of adipose tissue and weight gain leading to an unesthetic change in the figure.

The active agent or the combination of active agents, when it is suitable for oral administration, may in particular be used in a cosmetic composition intended for the oral administration of a food supplement.

The invention also relates to a cosmetic process for preventing and/or combating effects on adipose tissue, comprising the oral administration to an individual of petroselinic acid or of a combination of active agents in accordance with the invention.

The invention is also directed toward a cosmetic process for combating cellulite, comprising the oral administration to an individual of petroselinic acid or of a combination of active agents in accordance with the invention.

The invention also relates to a cosmetic process for combating the signs of modification of the skin surface during an upward or downward variation in weight, comprising at least the oral administration to an individual of petroselinic acid or of a combination in accordance with the invention.

The processes according to the invention have the characteristics of cosmetic processes insofar as they make it possible to improve the attractiveness of the figure, in particular by combating cellulite and unsightly accumulations of subcutaneous adipose tissue. In addition, a combination of active agents, a composition or a food supplement according to the invention may be used daily for several months, without a medical prescription. The present invention thus clearly lies outside the therapeutic field.

The invention also relates to a food supplement comprising one part of the compounds comprising petroselinic acid or forming the combination in accordance with the invention in a first composition, and at least the other part of the compounds forming the combination in a second composition, as a kit or combination product for simultaneous use, separate use or sequential use over time.

It is understood in the context of the present invention that “the oral cosmetic use” covers the use of products administered orally, these products being, for example, in the form of a food supplement as outlined below. These products produce an esthetic and comfort effect on the skin, or else an effect for beauty purposes, for example for the purposes of combating cellulite, unsightly accumulations of subcutaneous adipose tissue, and any sign of modification of the skin surface during an upward or downward variation in weight.

In the context of the present invention, the term “viscoelastic or biomechanical properties of the skin” means the extensibility, tonicity, firmness, suppleness and/or elasticity properties of the skin.

The expression “esthetic disorders of the figure” means skin disorders induced by weight-loss and/or slimming diets and skin disorders induced by cellulite.

The expression “skin disorders induced by weight-loss and/or slimming diets” means all the modifications of the external appearance of the skin, for instance the flaccid skin appearance that may be more or less marked following weight loss.

The expression “skin disorders induced by cellulite” means all the modifications of the external appearance of the skin, for instance the nodes of fat or “orange peel” which may be more or less localized in the areas of excess weight, such as the thighs, the arms or the abdomen.

The term “preventing” is intended to mean “reducing the risk of developing”.

Unless otherwise indicated, the term “treating” means any action directed toward improving the comfort or well-being of an individual; this term therefore covers both the notions of attenuating or relieving and curing.

The weight modification, and in particular weight loss, may be due to a slimming diet or to pregnancy.

The cosmetic surgery procedure may be chosen from lifting, liposuction and the injection of filling products.

### COMBINATION OF ACTIVE AGENTS

a) Petroselinic Acid

According to a first embodiment variant, petroselinic acid, (C18:1 n-12 or cis delta 6) monounsaturated fatty acid or C18 delta-6-cis-octadecenoic acid, may be used in an isolated form.

According to another variant of the invention, petroselinic acid is used in the form of a plant extract containing same, such as an oil. This form is particularly suitable for oral administration.

The petroselinic acid-rich oils are more particularly chosen from umbellifera plant oils.

The expression “petroselinic acid-rich oil” means an oil comprising at least 40% of petroselinic acid.

Umbellifera plants are plants whose flowers are arranged in umbels, and the species that are particularly rich in petroselinic acid are Umbellifarea-Apiacea and Araliaceae. Plants of the Thapsia genus are also sources of petroselinic acid (Avato et al., Lipids, 2001, 36, 845).

The species preferably used in the invention are coriander, chervil, carrot, celery, cumin, caraway, parsley and dill, or mixtures thereof. The petroselinic acid-source umbellifera plant oil that is most particularly suitable for use in the invention may be extracted from the seed of these umbellifera plants, for example by milling or pressing, and then refining.

The umbellifera plant oil has a petroselinic acid content which varies according to the umbellifera plant seed from which it is extracted. For the same umbellifera plant, the petroselinic acid content also varies according to the country of origin of the umbellifera plant and according to the extraction, which may be more or less complete.

Petroselinic acid is also an abundant compound (approximately 48%) of Geranium sanguineum seed oil.

In particular, petroselinic acid may be used in the form of an umbellifera plant oil or Geranium sanguineum oil.

Thus, according to one embodiment, the umbellifera plant oil more particularly considered in the invention may be chosen from dill, parsley, caraway, cumin, celery, carrot, chervil and coriander seed oils, and mixtures thereof.

Preferably, petroselinic acid is used in the form of a coriander seed oil. According to the present invention, coriander seed oils are covered by the expression “coriander oil”.

The contents are variable depending on whether the combination of active agents in accordance with the invention is used in a cosmetic composition intended for oral administration via a food supplement.

According to one embodiment, petroselinic acid or a combination of active agents according to the invention is used in a food supplement.

The petroselinic acid content, in a cosmetic composition intended for oral administration or in a food supplement, in accordance with the invention, may be between 0.01% and 70% by weight, especially between 0.1% and 70% by weight, and particularly between 1% and 70% by weight, relative to the total weight of the composition or of the food supplement.

The petroselinic acid content in a cosmetic composition intended for oral administration or a food supplement, in accordance with the invention, may be such that the daily dose of said petroselinic acid is between 0.5 and 2000 mg/day, particularly between 1 and 1000 mg/day, and especially between 5 and 700 mg/day.

b) Lycopene

A combination of active agents according to the invention may also comprise lycopene.

Lycopene is a natural pigment found in ripe fruit, particularly in tomato, but it also exists in synthetic form, especially synthesized from a fungus, Blakeslea trispora.

It belongs to the carotenoid family and its structure is similar to that of β-carotene.

It may in particular be sold by the company Lycored under the name Lyc-O-Mato®.

Preferably, lycopene is used in a combination of active agents in accordance with the invention. In other words, according to one embodiment, the combination of active agents comprises, or even consists of, petroselinic acid and lycopene.

The lycopene content in a cosmetic composition intended for oral administration or a food supplement, in accordance with the invention, may be such that the daily dose of lycopene is between 0.01 and 20 mg/day, particularly between 0.1 and 15 mg/day, and especially between 0.5 and 10 mg/day.

c) Taurine

A combination of active agents according to the invention may comprise taurine, or hypotaurine. It may also use a salt thereof. The salts that may be used are obviously chosen for their total harmlessness. Alkali metal or alkaline-earth metal salts, in particular magnesium salts, manganese, iron(II) or zinc salts are suitable in this respect.

The content of taurine, hypotaurine or a salt thereof in a cosmetic composition intended for oral administration or a food supplement in accordance with the invention may be such that the daily dose of said taurine, hypotaurine or a salt thereof is between 1 and 700 mg/day, particularly between 10 and 500 mg/day and especially between 50 and 300 mg/day.

d) Zinc

The term “zinc” means zinc or a salt thereof (zinc acetate, chloride, citrate, lactate, gluconate, lactate, oxide, carbonate or sulfate), in particular Zn(II) salts, preferably complexed with one or more (poly)hydroxy acids such as gluconate.

The term “(poly)hydroxy acid” means any carboxylic acid which comprises a hydrocarbon-based chain which is linear or branched, and saturated or unsaturated, preferably saturated and/or linear, comprising from 1 to 10 carbon atoms and from 1 to 9 hydroxyl groups, and comprising from 1 to 4 carboxylic groups —C(O)—OH, at least one of said —C(O)—OH functions of which is in the carboxylate form —C(O)—O— complexed with the Zn atom, preferably Zn(II).

More particularly, the zinc salt is complexed with two carboxylate groups such as that of formula (I):

R—C(O)—O—Zn—O—C(O)—R′ (I)

in which R and R′, which may be identical or different, represent a (C1-C6) (poly)hydroxyalkyl group, and also solvates thereof, such as hydrates, and enantiomers thereof.

Preferably, the compound of formula (I) is zinc gluconate.

According to a particular embodiment of the invention, the zinc is not a zinc oxide, but a zinc salt. The term “Zn(II)” means a zinc atom in oxidation state Zn²⁺.

The content of zinc gluconate in a cosmetic composition intended for oral administration or in a food supplement in accordance with the invention may be such that the daily dose of said zinc gluconate is between 0.01 and 300 mg/day, especially between 0.1 and 200 mg/day, and in particular between 1 and 100 mg/day.

The active agent or the combination of active agents in accordance with the present invention may also be used with additional active agents suitable for the mode of administration considered, as is described hereinbelow.

The active agent or the combination of active agents in accordance with the invention may be used with additional active agents chosen from (i) lipolysis modulators, for instance methylxanthines (caffeine, theophylline, aminophylline) or phosphatidylcholine and also hormones and extracts of plants such as guarana or konjac, or (ii) inflammation modulators, for instance hesperidin, Omega 3 and Omega 6 polyunsaturated fatty acids, gamma-linoleic acid and oils comprising same, such as echium oil, vitamins B3 niacin/niacinamide and B8, anti-inflammatory peptides, for instance KPV (Lysine Proline Valine) and vitamin D3.

According to one embodiment, a cosmetic composition for oral administration or a food supplement in accordance with the invention may also comprise at least one vitamin chosen from vitamin B1, B3, B5, B6, B8, B12, C, D, and especially D3, or PP, tocopherol (vitamin E) and derivatives thereof, especially an ester such as tocopheryl acetate or palmitate, preferably tocopheryl acetate.

According to this embodiment, a cosmetic composition for oral administration or a food supplement in accordance with the present invention preferably comprises at least vitamin E or a derivative thereof and/or vitamin D, preferentially vitamin E or a derivative thereof and vitamin D, preferentially vitamin D3 and tocopheryl acetate.

Thus, according to a preferred embodiment of the invention, the present invention is directed toward a cosmetic composition intended for oral administration or a food supplement comprising petroselinic acid, taurine, zinc, preferably zinc gluconate, vitamin D3 and tocopheryl acetate.

The compositions according to the invention may also comprise at least one carotenoid other than lycopene, especially a carotenoid chosen from β-carotene, astaxanthin, zeaxanthin and lutein, flavonoids such as catechins, hesperidin, proanthocyanidins, especially in the form of blackcurrant seed oil, anthocyanins, ubiquinones, coffee extracts containing polyphenols and/or diterpenes, chicory extracts, ginkgo biloba extracts, proanthocyanidin-rich grape extracts, pimento extracts, soybean extracts, other sources of flavonoids with antioxidant properties, fatty acids, prebiotics, probiotics, resveratrol, selenium amino acids and glutathione precursors.

The oral compositions or food supplements may also comprise at least one probiotic, a prebiotic or a mixture of probiotics and a mixture of prebiotics. As probiotic microorganisms, mention may be made especially of Lactobacillus johnsonii (LA1) or Lactobacillus paracaseï (ST11); Lactobacillus rhamnosus (LPR).

The cosmetic compositions intended for oral administration or food supplements in accordance with the present invention may be in any oral-route galenical form normally used.

According to one embodiment, a cosmetic composition intended for oral administration or a food supplement in accordance with the invention comprises:

(i) petroselinic acid in a content of between 1% and 70% by weight, especially between 10% and 70% by weight and particularly between 20% and 70% by weight relative to the total weight of the combination of active agents;

(ii) taurine in a content of between 1% and 50% by weight, especially between 5% and 40% by weight and particularly between 10% and 30% by weight relative to the total weight of the combination of active agents; and/or

(iii) at least one zinc (poly)hydroxy acid, preferably zinc gluconate, in a content of between 0.001% and 40% by weight, especially between 0.01% and 25% by weight and particularly between 0.1% and 20% by weight relative to the total weight of the combination of active agents;

(iv) optionally vitamin D3 in a content of between 0.0001% and 1.0% by weight, especially between 0.0001% and 0.5% by weight and particularly between 0.0001% and 0.1% by weight relative to the total weight of the combination of active agents; and

(v) optionally tocopheryl acetate in a content of between 0.01% and 10% by weight, especially between 0.1% and 10% by weight and particularly between 0.2% and 5% by weight relative to the total weight of the combination of active agents.

According to a particular embodiment, a cosmetic composition for oral administration or a food supplement in accordance with the invention comprises ingredients i) to v), taken together or individually:

(i) petroselinic acid in a content of between 1% and 70% by weight, especially between 10% and 70% by weight and particularly between 15% and 70% by weight relative to the total weight of the composition;

(ii) taurine in a content of between 1% and 40% by weight, especially between 5% and 40% by weight and particularly between 5% and 30% by weight relative to the total weight of the composition; and/or

(iii) at least one zinc (poly)hydroxy acid, preferably zinc gluconate, in a content of between 0.001% and 30% by weight, especially between 0.01% and 25% by weight and particularly between 0.1% and 20% by weight relative to the total weight of the composition;

(iv) optionally vitamin D3 in a content of between 0.0001% and 1.0% by weight, especially between 0.0001% and 0.5% by weight and particularly between 0.0001% and 0.1% by weight relative to the total weight of the composition; and

(v) optionally tocopheryl acetate in a content of between 0.01% and 10% by weight, especially between 0.1% and 10% by weight and particularly between 0.2% and 5% by weight relative to the total weight of the composition.

According to a particular embodiment, the cosmetic composition for oral administration or the food supplement comprises all of the abovementioned ingredients (i) to (iii).

According to a particular embodiment, the cosmetic composition for oral administration or the food supplement comprises all of the abovementioned ingredients (i) to (v).

For ingestion, numerous embodiments of oral compositions and especially of food supplements are possible. Their formulation is performed via the usual processes for producing coated tablets, gel capsules, gels, emulsions, tablets, lozenges or wafer capsules.

The compositions according to the invention, intended for oral administration, may especially comprise all or only a part of the daily dose.

In other words, one to three compositions may be administered per day.

Typically, the duration of this cosmetic treatment for oral administration may be greater than 4 weeks, especially from 4 to 15 weeks, with, where appropriate, one or more periods of stoppage which may range from a few days to several months.

The food supplement in accordance with the present invention may comprise one part of the active agents forming the combination according to the invention in a first composition, and the other part of these active agents in a second composition, as a kit or combination product for simultaneous use, separate use or sequential use over time.

This supplement may be formulated in such a way that the two compositions are in the same forms or in different forms, for example chosen from those mentioned above. Such a kit may in particular be provided in one and the same packaging.

The active agents according to the invention may be formulated with the usual excipients and components for oral compositions or food supplements according to the invention, i.e. especially fatty and/or aqueous components, humectants, thickeners, preserving agents, texture agents and/or coating agents, antioxidants and dyes that are common in the food sector.

As cosmetic active agent other than the combination of active agents in accordance with the invention, a cosmetic composition for oral administration or a food supplement may advantageously contain an additional active agent chosen from vitamin C, glucosamine, collagen and hyaluronic acid, and mixtures thereof.

Still as cosmetic active agent other than the combination of active agents in accordance with the invention, a cosmetic composition for oral administration or a food supplement may advantageously also comprise at least one inhibitor of matrix degradation, for instance extracts of rosemary, rosmarinic acid; carnosic acid, curcumin, pine bark extract; pycnogenol; berberin; Boswellia extracts; emodin; sesamol; sulforaphane and broccoli extracts; resveratrol and grape extracts; 6-shogaol; blackcurrant extracts; aubergine extracts; enterolactone; loquat extracts; olive extracts, for instance oleuropein; pachymic acid, pterostilbene, hydroxytyrosol, and mixtures thereof.

Still as cosmetic active agent other than the combination of active agents in accordance with the invention, a cosmetic composition for oral administration or a food supplement may advantageously also comprise anti-inflammatory probiotics and/or weight modulators, for instance Lactobacillus LGG, LPR ST11 or Lactobacillus johnsonii LA1.

Needless to say, those skilled in the art will take care to select the optional compound(s) to be added to the compositions in accordance with the invention and also the concentration thereof, such that the advantageous properties intrinsically associated with the compositions in accordance with the invention are not, or are not substantially, adversely affected by the envisaged addition.

Moreover, the administration of the combination of active agents in accordance with the present invention may advantageously be combined with radiofrequency procedures, draining procedures, the application of ultrasound, laser treatments and/or physical methods of massage such as endermology or liposuction according to techniques known to those skilled in the art.

Other characteristics and advantages of the invention will emerge more clearly from the examples that follow, which are given as non-limiting illustrations.

### EXAMPLE 1

Oral Composition in Soft Capsule Form

| Ingredients | (mg/soft capsule) |
|---|---|
| Coriander seed oil (of which 65% | 300.00 |
| of petroselinic acid) | |
| Taurine | 76.10 |
| Zinc gluconate | 25.75 |
| Vitamin E | 4.10 |
| Vitamin D3 | 0.115 |
| Excipients | |
| Refined coconut oil | 112.00 |
| Yellow beeswax, Cera flava | 22.000 |
| Sunflower lecithin | 10.00 |
| Capsule | |
| Fish gelatin | 144.6 |
| Glycerol | 58.6 |
| Purified water | 6.8 |

### EXAMPLE 2

Oral Composition as a Stick in Emulsion Form

| Ingredients | (g/stick) |
|---|---|
| Coriander seed oil | 0.65 |
| (of which 65% of petroselinic | |
| acid) | |
| Vitamin E | 0.0082 |
| Excipients | |
| Water | 1.722 |
| Sugar | 0.911 |
| Fructose | 0.911 |
| Microcrystalline cellulose | 0.032 |
| Sodium carboxymethylcellulose | 0.004 |
| Natural mixture of tocopherols | 0.034 |
| Sunflower oil | 3.015 |
| Natural lemon flavoring | 0.034 |
| Potassium sorbate | 0.013 |
| Citric acid | 0.013 |
| Propylene glycol alginate | 0.010 |

### EXAMPLE 3

Demonstration of the Stimulatory Effect of Petroselinic Acid and of the Combination of Petroselinic Acid with Lycopene According to the Invention on the Synthesis/basal Release of Lipoxin A4 by Keratinocytes

Mononuclear blood cells are cultured under 5% CO₂ and at 37° C. in a serum-free medium for macrophages (SFM Macrophage; Invitrogen 12065074) for 24 hours.

After this step, the medium is replaced with the same fresh test medium also containing the active agents at the various doses for 30 minutes in the presence of the various products to be evaluated, as indicated in the table of results below. The inflammatory response was then triggered in the presence of phorbol myristate (0.05 μM) and calcium ionophore (1 μM) and of a lipid substrate mixture composed of docosahexaenoic acid (DHA—1 μg/mL) and eicosapentaenoic acid (EPA—1 μg/mL).

The supernatants were then collected after 2 hours of stimulation and frozen at −80° C. before preparation for analysis by mass spectrometry.

Experimental triplicates (three wells) were prepared per experimental condition. Into each culture plate was placed a control corresponding to cells stimulated with the PMA/A23187 mixture and/or with addition of the equimolar mixture of fatty acids.

The thawed supernatants were concentrated by solid-phase extraction (SPE) and taken up in methanol before spectrometric analysis. The analytical method used consists in separating the various analytes by high-pressure liquid chromatography as a function of their retention time and in quantifying them by mass spectrometry.

The analyses were performed using an LC 1290 Infinity chain (Agilent Technologies) coupled to a 6460 Triple Quad LC/MS mass spectrometer (Agilent Technologies) equipped with an electrospray ionization source (Jet stream technology) operating in negative mode. The chromatographic separations were performed on a ZorBAX SB-C18 column.

The results were obtained in pg/mL of cell supernatant. These raw data were then transformed by calculation to obtain the percentage of activation (or of inhibition) of the plate relative to the control using the following calculation:

% modulation=100×(value obtained with the active agent−value of the control)/value of the control

These percentages of modulation are reported in the table of results below.

A combination of active agents in accordance with the invention comprising coriander oil, rich in petroselinic acid, and Lycomato, rich in lycopene, and also these same compounds individually, were tested in accordance with that indicated above.

The results obtained after these comparative tests are as follows:

| | Level of lipoxin A4 |
|---|---|
| Compounds tested | production |
| Coriander oil | +19% |
| (of which between 60% and 75% | |
| of petroselinic acid) | |
| 0.25 mg/ml | |
| Lycomato (containing 10% of | +0% |
| lycopene) | |
| 0.001 mg/ml | |
| Lycomato (containing 10% of | +90% |
| lycopene) | |
| 0.001 mg/ml + coriander oil | |
| (of which between 60% and 75% | |
| of petroselinic acid) 0.25 mg/ml | |

It is observed that coriander oil containing petroselinic acid stimulates the production of lipoxin A4.

Lycomato alone does not, itself, lead to any variation in the level of production of lipoxin A4.

However, it may be seen that the effect of a combination in accordance with the invention on the production of lipoxin A4 is very markedly greater than the sum of the effects of the compounds used individually.

Specifically, an increase in the production of lipoxin A4 of 90% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

### EXAMPLE 4

Demonstration of the Stimulatory Effect of Petroselinic Acid and of the Combination of Petroselinic Acid with Taurine According to the Invention on the Synthesis/basal Release of Lipoxin A4 by Keratinocytes

A protocol similar to that described in Example 3 was performed, replacing, however, the Lycomato with taurine.

Thus, a combination of active agents in accordance with the invention comprising coriander oil, rich in petroselinic acid, and taurine, and also these same compounds individually, were tested in accordance with that indicated below.

The results obtained after these comparative tests are as follows:

| | Level of lipoxin A4 |
|---|---|
| Compounds tested | production |
| Coriander oil | +19% |
| (of which between 60% and 75% | |
| of petroselinic acid) | |
| 0.25 mg/ml | |
| Taurine | +38% |
| 3.1 mg/ml | |
| Taurine 3.1 mg/ml + coriander oil | +118% |
| (of which between 60% and 75% | |
| of petroselinic acid) 0.25 mg/ml | |

It is thus observed that coriander oil containing petroselinic acid stimulates the production of lipoxin A4.

In this case also, it may be seen that the effect of a combination in accordance with the invention on the production of lipoxin A4 is very markedly greater than the sum of the effects of the compounds used individually.

Specifically, an increase in the production of lipoxin A4 of 118% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

### EXAMPLE 5

A protocol similar to that described in Example 3 was performed, replacing, however, the Lycomato with zinc gluconate.

Thus, a combination of active agents in accordance with the invention comprising coriander oil, rich in petroselinic acid, and zinc gluconate, and also these same compounds individually, were tested in accordance with that indicated below.

The results obtained after these comparative tests are as follows:

| | Level of lipoxin A4 |
|---|---|
| Compounds tested | production |
| Coriander oil | +19% |
| (of which between 60% and 75% of | |
| petroselinic acid) | |
| 0.25 mg/ml | |
| Zinc gluconate | −5% |
| 0.005 mg/ml | |
| Zinc gluconate 0.005 mg/ml + coriander oil | +94% |
| (of which between 60% and 75% of | |
| petroselinic acid) 0.25 mg/ml | |

In this case also, it may be seen that the effect of a combination in accordance with the invention on the production of lipoxin A4 is very markedly greater than the sum of the effects of the compounds used individually.

Specifically, an increase in the production of lipoxin A4 of 94% relative to the basal level of production of this anti-inflammatory component could be seen when the lymphocyte cells tested were placed in contact with the combination of active agents.

It is thus indeed a synergistic effect of a combination of active agents in accordance with the invention that is observed and demonstrated here.

### EXAMPLE 6

A composition such as a food supplement or an oral composition in accordance with the invention may in particular have the following contents:

| | % by weight relative to the total |
|---|---|
| Components | weight of the composition |
| Petroselinic acid | 54.9 (provided by the coriander seed |
| | oil) |
| Zinc gluconate | 6.3 (of which 13.6% of active |
| | material) |
| Taurine | 18.7 (of which 98.5% of active |
| | material) |
| Vitamin E | 1.0 (of which 67% of active material) |
| Vitamin D3 | 0.03 (of which 2.5% of active |
| | material) |

1-15. (canceled)

16. A cosmetic method for combating aesthetic disorders of the figure associated with adipose tissue modifications comprising at least one step consisting of an oral administration of petroselinic acid.

17. The method as claimed in claim 16 wherein that petroselinic acid is combined with at least one compound chosen from taurine, zinc, a salt thereof, lycopene, and mixtures thereof.

18. The method as claimed in claim 16 wherein that petroselinic acid is combined with at least taurine or zinc glutonate or combined with at least taurine and zinc glutonate.

19. A cosmetic method for limiting the expansion of and/or reducing adipose tissue and/or reducing subcutaneous adipose tissue comprising at least one step consisting of an oral administration of petroselinic acid, or of a combination of active agents comprising at least petroselinic acid and at least one compound chosen from taurine, zinc, a salt thereof, lycopene, and mixtures thereof.

20. A cosmetic method for limiting the expansion of and/or reducing adipose tissue and/or reducing subcutaneous adipose tissue comprising at least one step consisting of an oral administration of a combination of active agents comprising at least petroselinic acid and at least taurine or zinc glutonate or comprising at least petroselinic acid and at least taurine and zinc glutonate.

21. The method as claimed in claim 16, for reducing the weight of fat mass of an individual.

22. The method as claimed in claim 16, for reducing the total weight of an individual.

23. The method as claimed in claim 16, for improving the figure by limiting or reducing the unsightly accumulations of subcutaneous adipose tissue on the waist, the hips, the thighs, the tummy, the arms and the face.

24. The method as claimed in claim 16, for combating cellulite.

25. The method as claimed in claim 16, for preventing and/or treating the visible manifestations of cellulite and/or the orange peel appearance of the skin and/or else for treating stretch marks or for preventing their appearance.

26. The method as claimed in claim 16, for combating the signs of modification of the skin surface during an upward or downward variation in weight, during weight loss observed in the course of a slimming diet or during weight loss observed prior to, simultaneously with and/or subsequent to a cosmetic surgery procedure.

27. The method as claimed in claim 16, for maintaining and/or restoring the biomechanical properties of the skin, chosen from the extensibility, tonicity, firmness, suppleness, density and/or elasticity properties of the skin.

28. The method as claimed in claim 16, in which said petroselinic acid is used in an isolated form or in the form of a plant extract containing same.

29. The method as claimed in claim 16, in which said petroselinic acid is in the form of an oil.

30. The method as claimed in claim 16, in which said petroselinic acid is used in the form of an umbellifera plant oil or Geranium sanguineum oil.

31. The method as claimed in claim 30, in which said umbellifera plant oil is chosen from dill, parsley, caraway, cumin, celery, carrot, chervil and coriander seed oils, and mixtures thereof.

32. The method as claimed in claim 16, in which the combination of active agents is used in a food supplement.

33. A cosmetic process for preventing and/or combating effects on adipose tissue, comprising the oral administration to an individual of petroselinic acid, or of a combination of active agents comprising at least petroselinic acid and at least one compound chosen from zinc, taurine, a salt thereof, lycopene, and mixtures thereof.
